(19) United States
(12) Patent Application Publication
(10) Pub. No.: US 2026/0090847 A1
(43) Pub. Date: Apr. 2, 2026

(54) SURGICAL ROBOTIC SYSTEMS FOR MICROWAVE WATERJET

(71) Applicants: BEIJING SHIJITAN HOSPITAL, CAPITAL MEDICAL UNIVERSITY, Beijing (CN); CHINA SCIENCE HUACHEN INNOVATECH (ZHONGKE HUASHEN, BEIJING) CO., LTD., Beijing (CN)

(72) Inventors: Sidan LI, Beijing (CN); Yongbing CHEN, Beijing (CN); Qian ZHANG, Beijing (CN)

(73) Assignees: BEIJING SHIJITAN HOSPITAL, CAPITAL MEDICAL UNIVERSITY, Beijing (CN); CHINA SCIENCE HUACHEN INNOVATECH (ZHONGKE HUASHEN, BEIJING) CO., LTD., Beijing (CN)

(21) Appl. No.: 19/178,811

(22) Filed: Apr. 14, 2025

(30) Foreign Application Priority Data
Jul. 1, 2024 (CN) 202410874288.9

(51) Int. Cl. A61B 34/30; A61B 17/3203

(52) U.S. Cl. CPC A61B 34/30; A61B 17/3203; A61B 2034/305

(57) ABSTRACT

A surgical robotic system for microwave waterjet is provided, including a cutting blade, a serpentine joint pipe, a mechanical arm main body, a workbench and a medical booster pump. The cutting blade is connected to the mechanical arm main body through the serpentine joint pipe, the mechanical arm main body is electrically connected to the workbench, and connected to the medical booster pump via a pipeline, the cutting blade includes a casing, a microwave blade, a cooling support, a high-pressure jet pipe and a return pipe, a front end of the microwave blade is provided with a curved cutting edge, and a rear end of the microwave blade is provided in the cooling support, a jet hole is provided on one side of the curved cutting edge, the high-pressure jet pipe is connected with the jet hole, and the return pipe is provided on one side of the microwave blade.

### CROSS-REFERENCE TO RELATED APPLICATION

This application claims priority of Chinese Patent Application No. 202410874288.9, filed on Jul. 1, 2024, the content of which is entirely incorporated herein by reference.

### TECHNICAL FIELD

The present disclosure relates to the field of microwave waterjet technology, and in particular, to a surgical robotic system for microwave waterjet.

### BACKGROUND

A surgical robot is an advanced medical device invented with the help of microtrauma surgery and development of related underlying technologies. The surgical robot is a segment of a medical robot. The surgical robot is a precisely operated robot that surgeons can control remotely and operate precisely into a patient's body through tiny incisions, thereby assisting the surgeons in performing minimally invasive surgery. The surgical robot is used in a field of minimally invasive surgeries beyond human capabilities, to achieve precise operations that fail to be accomplished by humans alone. A laparoscopic surgical robot typically consists of a surgical console, a surgical vehicle equipped with a robotic arm, and a visualization system. The robotic arm simulates a human arm which provides the surgeons with a series of motions that simulates those of a human wrist while filtering out tremors of a human hand. The most mature kind of current laparoscopic surgical robots is a DaVinci surgical robot, which has transformed the field of minimally invasive surgery for more than 20 years with a platform that has pioneered new capabilities in an operating room. With more than 5 million surgeries performed, Intuitive has become a recognized leader in the surgical robotics. A strength of DaVinci surgical robot lies in that a distal end of the mechanical arm of the DaVinci surgical robot is able to flexibility rotate. But unfortunately at present, both the DaVinci and other robots lack energy apparatus, for example, although a waterjet is able to be softly connected to soft tissues of the human body for a rapid and accurate cutting, a hemostasis function of the waterjet is weak, and an impact caused by water flow is also not conducive to a rapid hemostasis.

There is therefore an urgent need to design a surgical robotic system for microwave waterjet combined with a microwave hemostat to combine advantages of a precise cutting and a good hemostasis.

### SUMMARY

One or more embodiments of the present disclosure provide a surgical robotic system for microwave waterjet. The system includes a cutting blade, a serpentine joint pipe, a mechanical arm main body, a workbench, and a medical booster pump. The cutting blade is connected to the mechanical arm main body through the serpentine joint pipe, the mechanical arm main body is electrically connected to the workbench, and the mechanical arm main body is connected to the medical booster pump via a pipeline, the cutting blade includes a casing, a microwave blade, a cooling support, a high-pressure jet pipe, and a return pipe, and each of two sides of a front end of the casing is provided with a protective plate, a front end of the microwave blade is provided with a curved cutting edge, the curved cutting edge is located at the front end of the casing and is located between the two protective plates, and a rear end of the microwave blade is provided in the cooling support, the casing is filled with a filler for fixing the cooling support, a tail end of the microwave blade is connected with a microwave conductor line, a jet hole is provided on one side of the curved cutting edge, the high-pressure jet pipe passes through the cooling support and is connected with the jet hole, and the return pipe passes through the cooling support and is provided on one side of the microwave blade.

In some embodiments, the cooling support includes an annular support and thermal insulation boards which are spaced apart from each other, the thermal insulation boards are provided in the annular support, the thermal insulation boards are provided with an isolation mounting groove, a first mounting hole, and a second mounting hole along a direction of the annular support, the rear end of the microwave blade is mounted in the isolation mounting groove, thermal conductive silicone is provided between the thermal insulation boards in the isolation mounting groove, the high-pressure jet pipe is provided in the first mounting hole, the return pipe is provided in the second mounting hole, and the filler is located between the thermal insulation boards that are spaced apart in each layer.

In some embodiments, an end of the isolation mounting groove is provided with a sealing ring for sealing the thermal conductive silicone.

In some embodiments, a cable shielding layer and an isolation layer are provided on a surface of the microwave conductor line sequentially from inside to outside.

In some embodiments, the jet hole coincides with an axis of the high pressure jet pipe.

In some embodiments, the serpentine joint pipe includes a flexible inner support pipe and a plurality of connecting pipe units fixed to the flexible inner support pipe at intervals, and the high pressure jet pipe, the return pipe, and the microwave conductor line are extended to an interior of the flexible inner support pipe, a front end of the flexible inner support pipe is fixedly connected to the casing, a rear end of the flexible inner support pipe is fixedly connected to the mechanical arm main body, a front end of each connecting pipe unit and a front end of the mechanical arm main body are symmetrically provided with two circular lugs, a rear end of the each connecting pipe unit and a rear end of the casing are symmetrically provided with two circular grooves cooperating with the two circular lugs, a line connecting the two circular lugs on the same connecting pipe unit is perpendicular to a line connecting the two circular grooves, and connectors and the connecting pipe units, two adjacent connecting pipe units, and the connecting pipe units and the mechanical arm main body are all spliced to each other by the circular lugs and the circular grooves.

In some embodiments, a plurality of traction ropes are fixedly connected along a circumferential direction to a rear end face of the casing, and a periphery of each of the connecting pipe units as well as a periphery of the mechanical arm main body are provided with a threading hole at a position corresponding to each of the traction ropes, and one end of each of the traction ropes away from the casing passes through the threading holes in the connecting pipe unit and in the mechanical arm main body sequentially.

In some embodiments, the flexible inner support pipe is a rubber pipe.

### BRIEF DESCRIPTION OF THE DRAWINGS

The present disclosure will be further illustrated by way of exemplary embodiments, which is described in detail by means of the accompanying drawings. These embodiments are not limiting, and in these embodiments, the same numbering denotes the same structure, wherein:

FIG. 1 is a front view of a surgical robotic system for microwave waterjet according to some embodiments of the present disclosure;

FIG. 2 is a front section view of a cutting blade according to some embodiments of the present disclosure;

FIG. 3 is a section view in an A-A direction in FIG. 1; and

FIG. 4 is a three-dimensional (3D) view of a cooling support according to some embodiments of the present disclosure.

In the figures:
101—casing, 102—microwave blade, 103—high-pressure jet pipe; 104—return pipe; 105—filler; 106—microwave conductor line; 107—jet hole; 108—annular support; 109—thermal insulation board; 110—isolation mounting groove; 111—first mounting hole; 112—second mounting hole; 113—sealing ring; 201—flexible inner support pipe; 202—connecting pipe unit; 203—traction rope; 204—threading hole; 3—mechanical arm main body.

### DETAILED DESCRIPTION

To more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for those skilled in the art to apply the present disclosure to other similar scenarios according to these drawings without creative labor. Unless obviously obtained from the context or the context illustrates otherwise, the same numeral in the drawings refers to the same structure or operation.

It should be understood that the terms “system,” “device,” “unit,” and/or “module” as used herein is a way to distinguish between different components, elements, parts, sections or assemblies at different levels. However, the words may be replaced by other expressions if other words accomplish the same purpose.

As shown in the present disclosure and the claims, unless the context clearly suggests an exception, the words “a,” “an, ““one,” and/or “the” do not refer specifically to the singular, but may also include the plural. Generally, the terms “including” and “comprising” suggest only the inclusion of clearly identified steps and elements. In general, the terms “including” and “comprising” only suggest the inclusion of explicitly identified steps and elements that do not constitute an exclusive list, and the method or device may also include other steps or elements.

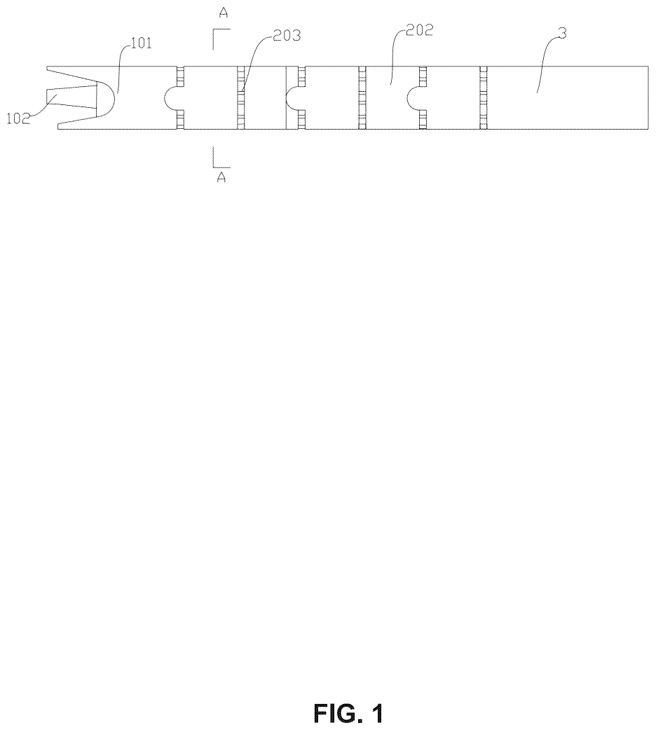

FIG. 1 is a front view of a surgical robotic system for microwave waterjet according to some embodiments of the present disclosure.

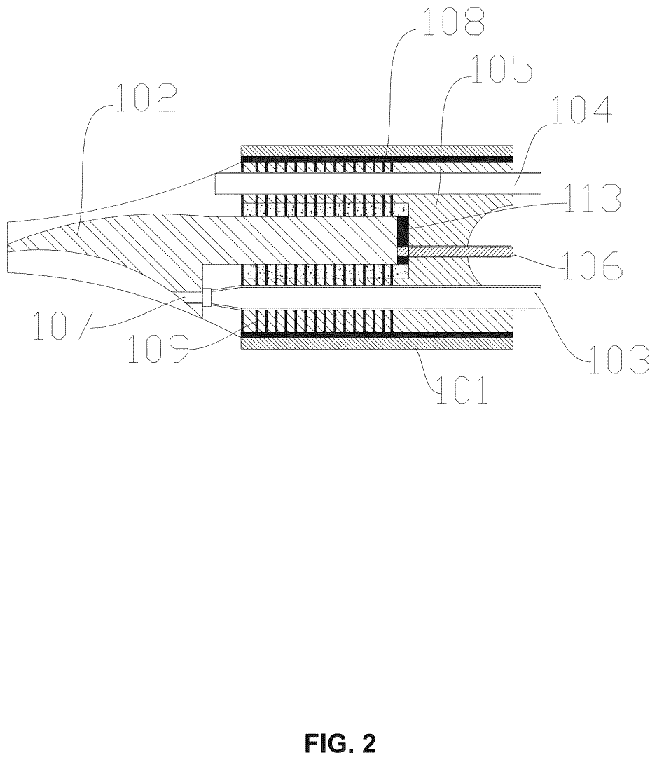

In some embodiments, as shown in FIG. 1, the surgical robotic system for microwave waterjet includes a cutting blade, a serpentine joint pipe, a mechanical arm main body 3, a workbench, and a medical booster pump. The cutting blade is connected to the mechanical arm main body 3 through the serpentine joint pipe. The mechanical arm main body 3 is electrically connected to the workbench, and the mechanical arm main body 3 is connected to the medical booster pump via a pipeline. The workbench and the mechanical arm main body 3 in the embodiment adopt existing devices, which is not repeated, and the workbench controls the mechanical arm main body 3 and the serpentine joint pipe, so as to realize a precise motion of the cutting blade, and to ensure that surgery is performed smoothly. More contents on the cutting blade and the serpentine joint pipe may be seen in FIGS. 2-4 and the related descriptions.

The cutting blade refers to a component used to cut soft tissues of the human body with a rapid hemostasis function.

FIG. 2 is a front section view of a cutting blade according to some embodiments of the present disclosure.

In some embodiments, as shown in FIG. 2, the cutting blade includes a casing 101, a microwave blade 102, a cooling support, a high-pressure jet pipe 103, and a return pipe 104. Each of two sides of a front end of the casing 101 is provided with a protective plate, a front end of the microwave blade 102 is provided with a curved cutting edge, the curved cutting edge is located at the front end of the casing 101 and is located between the two protective plates, and a rear end of the microwave blade 102 is provided in the cooling support, the casing 101 is filled with a filler 105 for fixing the cooling support, a tail end of the microwave blade 102 is connected with a microwave conductor line 106, a jet hole 107 is provided on one side of the curved cutting edge, the high-pressure jet pipe 103 passes through the cooling support and is connected with the jet hole 107, and the return pipe 104 passes through the cooling support and is provided on one side of the microwave blade 102. The front end refers to an end away from an operator, and the rear end refers to an end close to the operator.

The casing 101 refers to a component that sleeves on an outer side of the cutting blade to protect the cutting blade. In some embodiments, the protective plate is provided on each side of the front end of the casing 101.

The protective plate is a component that avoids the cutting blade from cutting into non-surgical parts of a human tissue. In some embodiments, the protective plate is two projections left after two U-shaped grooves are gouged from a front part of the casing 101.

The microwave blade 102 is a core component of the cutting blade used to cut soft tissues of a human body with a rapid hemostasis function. In some embodiments, a front end of the microwave blade 102 is provided with a curved cutting edge. The curved cutting edge is located at the front end of the casing 101 and is located between the two protective plates. A rear end of the microwave blade 102 is provided in a middle of the cooling support, the casing is filled with the filler 105 for fixing the cooling support.

The filler 105 refers to a filler material used to fix components such as the cooling support, the microwave blade 102, etc. In some embodiments, the filler 105 includes an epoxy adhesive. Notably, the filler 105 may be made of any other feasible materials, such as a urethane resin, a polyester resin, etc.

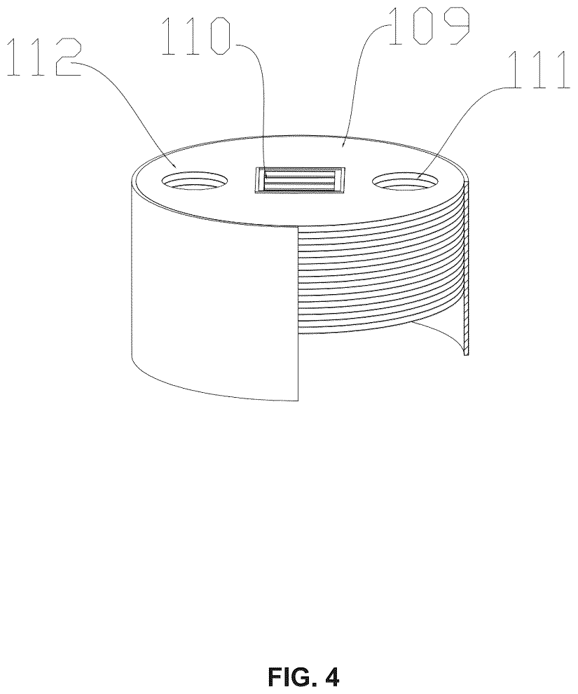

In some embodiments, the filler 105 is filled in the casing 101, after the filler 105 is cured, the filler 105 may fix the microwave blade 102 in the cooling support and has an insulating effect. More about the cooling support may be found in the relevant descriptions later (e.g. FIG. 4).

It should be noted that the microwave blade 102 may also be fixed in the cooling support in any other feasible manners, for example, bondings, snap connections, etc.

The microwave conductor line 106 may be used to transmit a microwave energy from an external source, such as a microwave generator, to the microwave blade 102 for a precise control of the microwave energy and a surgical manipulation.

In some embodiments, a cable shielding layer and an isolation layer are provided on a surface of the microwave conductor line 106 sequentially from inside to outside. The cable shielding layer and the isolation layer may bend and deform with the serpentine joint pipe.

The cable shielding layer refers to a shielding layer used to prevent external electromagnetic interferences from entering an interior of the cable, and at the same time to prevent the microwave energy inside the cable from radiating outward. The isolation layer refers to a shielding layer for isolating the interior of the cable from an external environment. For example, the isolation layer prevents environmental factors, such as moisture and dust, from affecting the shielding layer and the internal conductors of the cable.

Understandably, an external electromagnetic interference may be effectively prevented by disposing the cable shielding layer, and at the same time, the internal microwave energy may be prevented from leaking out of the cable, so as to ensure a stable transmission of the microwave energy. By disposing the isolation layer, an external mechanical damage on the microwave conductor line 106 is reduced, thereby extending a service life of the microwave conductor line 106, which in turn is conducive to an improvement of the stability and reliability of a microwave energy transmission.

The jet hole 107 is an aperture provided on one side of the curved cutting edge of the microwave blade 102. The jet hole 107 may be used to allow a jet of solution for cutting the soft tissues of the human body to pass through. The solution may include saline, etc.

In some embodiments, the jet hole 107 coincides with an axis of the high-pressure jet pipe 103. In some embodiments, the solution (e.g., the saline), after being pressurized by a medical booster pump, flows pass the high-pressure jet pipe 103 and is ejected through the jet hole 107 to cut the soft tissues, etc., of the human body.

The high pressure jet pipe 103 refers to a pipe that directs a flow of the high pressure solution to the jet hole 107. In some embodiments, the high-pressure jet pipe 103 is adapted to and connected to the jet hole 107 by progressively decreasing its own pipe diameter, as shown in FIG. 2.

In some embodiments of the present disclosure, by making the injection hole 107 coincide with the axis of the high-pressure jet pipe 103, it is possible to ensure that the solution is injected at the greatest possible speed, so as to reduce a loss of energy, and reduce a deviation phenomenon that occurs in the process of injection, thereby ensuring an accuracy of an ejection direction, and improving the accuracy and safety of the surgery.

In some embodiments, the solution after cutting is discharged through the return pipe 104, and the soft tissues after cutting is quickly hemostatic by the microwave blade 102

The return pipe 104 refers to a pipe for recycling the solution after cutting. In some embodiments, the return pipe 104 and the high-pressure jet pipe 103 are respectively provided on two sides of the microwave blade 102 through the cooling support, as shown in FIG. 2.

In some embodiments, a front portion of the return pipe 104 and the high-pressure jet pipe 103 that are disposed in the cooling support includes a rigid pipe body. The rigid pipe body allows the high-pressure jet pipe 103 and the return pipe 104 to facilitate a heat exchange while withstanding a high water pressure without deformation. It is appreciated that the remaining portions of the high pressure injection pipe 103 and the return pipe 104 are made of a flexible material (e.g., a rubber, etc.) to cooperate with a motion of the serpentine joint pipe.

It should be noted that sizes of the pipe diameters of the high-pressure jet pipe 103 and the return pipe 104 are set according to actual demands.

In some embodiments of the present disclosure, by combining the waterjet with the microwave blade 102, the solution is accelerated by using the medical booster pump. By using the solution ejected out from the jet hole 107 to quickly and accurately excise the soft tissues, a surgical excision time is greatly shortened. Moreover, the disposing of the microwave blade 102 has a very good hemostatic effect, and the solution ejected out may avoid a scab formation on cut surfaces, which brings a better cutting and hemostatic effect.

The cooling support refers to a component used to support and fix the various components of the cutting blade.

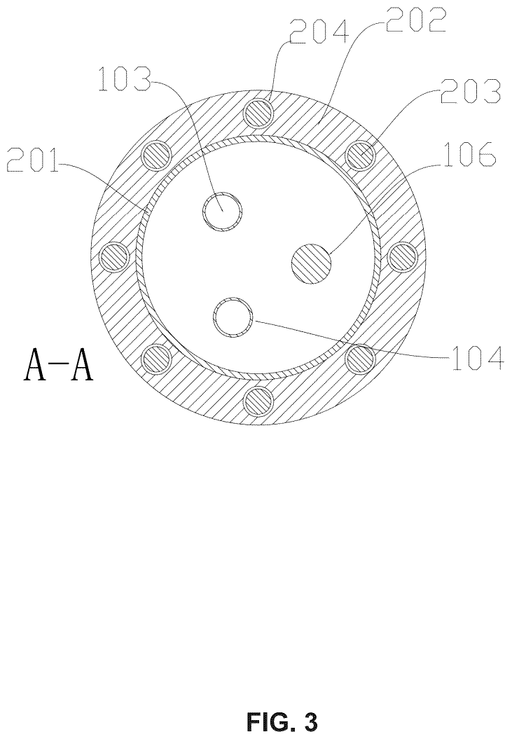

FIG. 3 is a section view in an A-A direction in FIG. 1. FIG. 4 is a three-dimensional (3D) view of a cooling support according to some embodiments of the present disclosure.

In some embodiments, as shown in FIGS. 2-4, the cooling support includes an annular support 108 and thermal insulation boards 109 which are spaced apart from each other. The thermal insulation boards 109 are provided in the annular support 108, the thermal insulation boards 109 are provided with an isolation mounting groove 110, a first mounting hole 111, and a second mounting hole 112 along a direction of the annular support 108. The rear end of the microwave blade 102 is mounted in the isolation mounting groove 110, thermal conductive silicone is provided between the thermal insulation boards 109 in the isolation mounting groove 110. The high-pressure jet pipe 103 is provided in the first mounting hole 111, the return pipe 104 is provided in the second mounting hole 112, and the filler 105 is located between the insulation boards 109 that are spaced apart in each layer.

The annular support 108 is a main frame of the cooling support. In some embodiments, the annular support 108 is annularly disposed within the casing 101 to ensure a structural stability of the cooling support.

The thermal insulation board 109 refers to a board structure for thermal conductivity and insulation. In some embodiments, the thermal insulation boards 109 are spaced apart in the annular support 108 along an axis direction of the casing 101, as shown in FIG. 2. The thermal insulation boards 109 may be designed in a variety of structural shapes, such as a circular plate shape.

The isolation mounting groove 110 is a groove-like structure provided on the thermal insulation boards 109. In some embodiments, the isolation mounting groove 110 provides a mounting position for the rear end of the microwave blade 102. The isolation mounting groove 110 is filled with the thermal conductive silicone. In some embodiments, heat generated during a working process of the microwave blade 102 is transferred well to the high-pressure jet pipe 103 and the return pipe 104 through the thermal conductive silicone using a thermal conductivity property of the thermal insulation boards 109, and then cooled down by using a solution (e.g., physiological saline) flowing through the high-pressure jet pipe 103 and the return pipe 104, so as to avoid that a temperature of the microwave blade 102 is too high and causes a thermal damage to an surgical region and the surrounding nerves and soft tissues.

In some embodiments, an end of the isolation mounting groove 110 is provided with a sealing ring 113 for sealing the thermal conductive silicone, as shown in FIG. 2.

The sealing ring 113 refers to an element used to seal the thermal conductive silicone from the external environment. In some embodiments, the sealing ring 113 is made of an elastic material, such as rubber or silicone. It is appreciated that by providing the sealing ring 113 at the end of the isolation mounting groove 110, it is possible to realize the sealing of the thermal conductive silicone while securing the connection between the microwave conductor line 106 and the microwave blade 102, to prevent a coolant or other media from entering an interior of the isolation mounting groove 110, thereby protecting the microwave blade 102 and the microwave conductor line 106 from a risk of corrosion or short circuit.

The first mounting hole 111 is an aperture-like structure provided on the thermal insulation boards 109. In some embodiments, the high-pressure jet pipe 103 is fixedly mounted in the first mounting hole 111 by the filler 105.

The second mounting hole 112 is another aperture-like structure provided on the thermal insulation boards 109. In some embodiments, the return pipe 104 is fixedly mounted in the second mounting hole 112 by the filler 105.

It is noted that structural dimensions (e.g., hole diameters) of the isolation mounting groove 110, the first mounting hole 111, and the second mounting hole 112 may be determined based on contour dimensions of the rear end of the microwave blade 102, the high-pressure jet pipe 103, and the return pipe 104, respectively.

In some embodiments of the present disclosure, the thermal insulation boards 109 stacked within the cooling support enable a sufficient heat exchange between the solution and the microwave blade 102 to realize a rapid cooling of the microwave blade 102, and avoid that the temperature of the microwave blade 102 is too high and causes the thermal damage to the surrounding nerves and soft tissues of the surgical region. In addition, by disposing the cooling support, the thermal insulation boards 109 in the isolation mounting groove 110 are used to support and position the microwave blade 102, which improves a structural strength of the cutting blade.

The serpentine joint pipe refers to a pipe structure in a shape of a snake. In some embodiments, the serpentine joint pipe is used to connect the cutting blade to the mechanical arm main body 3.

In some embodiments, as shown in FIG. 1 and FIG. 3, the serpentine joint pipe includes a flexible inner support pipe 201 and a plurality of connecting pipe units 202 fixed to the flexible inner support pipe 201 at intervals, and the high pressure jet pipe 103, the return pipe 104, and the microwave conductor line 106 are extended to an interior of the flexible inner support pipe 201, a front end of the flexible inner support pipe 201 is fixedly connected to the casing 101, a rear end of the flexible inner support pipe 201 is fixedly connected to the mechanical arm main body 3. In this embodiment, the high-pressure jet pipe 103 is connected to a medical booster pump through the mechanical arm main body 3 for a pressurized liquid supply to the cutting blade, and the return pipe 104 is connected to a negative pressure pumping device for a timely extraction of the solution. The microwave conductor line 106 is electrically connected to a microwave generator, a front end of the flexible inner support pipe 201 is fixedly connected to the casing 101, and a rear end of the flexible inner support pipe 201 is fixedly connected to the mechanical arm main body 3. By disposing the flexible inner support pipe 201, the plurality of connecting pipe units 202 are connected together without affecting the bending of the serpentine joint pipe.

The flexible inner support pipe 201 refers to a tubular structure with a certain degree of flexibility and supporting strength. In some embodiments, the flexible inner support pipe 201 is made of a variety of materials. For example, the flexible inner support pipe 201 is made of rubber or other flexible materials.

In some embodiments, the flexible inner support pipe 201 is a rubber pipe. Understandably, the usage of the rubber pipe for the flexible inner support pipe 201 not only ensures that the flexible inner support pipe 201 is flexibly bent and moved during a surgical procedure to adapt to different surgical operation needs; but also reduces a mechanical stress to which internal components are subjected when the serpentine joint pipe is bent, and prevents the components such as the high-pressure jet pipe 103, the reflux pipe 104, and the microwave conductor line 106 from being worn or damaged due to frequent bending.

The connecting pipe unit 202 refers to a tubular structure with a certain rigidity. In some embodiments, the connecting pipe units 202 are made of a rigid material, such as a metal or a hard plastic.

In some embodiments, the connecting pipe units 202 are spaced apart and fixedly sleeved to an outer side of the flexible inner support pipe 201, as shown in FIG. 1. The plurality of connecting pipe units 202 are used to form a plurality of movable joints of the serpentine joint pipe, which enables the entire serpentine joint pipe to flexibly bend and move in a plurality of directions while maintaining a structural stability and precision.

In some embodiments, as shown in FIG. 1, a front end of each connecting pipe unit 202 and a front end of the mechanical arm main body 3 are symmetrically provided with two circular lugs, a rear end of the each connecting pipe unit 202 and a rear end of the casing 101 are symmetrically provided with two circular grooves cooperating with the two circular lugs, a line connecting the two circular lugs on the same connecting pipe unit 202 is perpendicular to a line connecting the two circular grooves, and connectors and the connecting pipe units 202, two adjacent connecting pipe units 202, and the connecting pipe units 202 and the mechanical arm main body are all spliced to each other by the circular lugs and the circular grooves.

In some embodiments of the present disclosure, by the combination of the flexible inner support pipe 201 and the connecting pipe units 202, a flexible motion capability, a stable structural support, a protection of internal components, a simplified assembly and maintenance, an improved surgical safety and other effects are implemented, thereby improving a performance and a reliability of the surgical robotic system for microwave waterjet, and also ensuring a smooth surgical process and patient safety. In addition, as the microwave conductor line 106 and the high-pressure jet pipe 103 may both be made of flexible materials, the disposing of the serpentine joint pipe of some embodiments in the present disclosure does not interfere with the motion of the serpentine joint, and thus enables the cutting blade with the microwave blade 102 to swing flexibly.

In some embodiments, as shown in FIG. 3, a plurality of traction ropes 203 are fixedly connected along a circumferential direction to a rear end face of the casing 101, and a periphery of each of the connecting pipe units 202 as well as a periphery of the mechanical arm main body 3 are provided with a threading hole 204 at a position corresponding to each of the traction ropes 203, and one end of each of the traction ropes 203 away from the casing 101 passes through the threading holes 204 in the connecting pipe unit 202 and in the mechanical arm main body 3 sequentially, and is connected to a pulling device. Specific structures of the serpentine joint pipe and the pulling device may be found in the serpentine joint structure of the pipe and the wire driving carrier module in the Chinese patent No. CN114587600A.

The traction rope 203 refers to a flexible component for controlling the motion of the serpentine joint pipe. The threading hole 204 refers to an aperture for the traction rope 203 to pass through. In some embodiments, a plurality of threading holes 204 are provided at the periphery of the connecting pipe unit 202 and at the periphery of the mechanical arm main body 3 to ensure that the traction rope 203 is able to pass through smoothly and secure both at the respective positions.

In some embodiments of the present disclosure, by controlling the pulling device to pull the corresponding traction rope 203, the serpentine joint pipe is made to bend according to a change in a length of the traction rope 203, so as to drive the surgical cutting blade disposed at the front end to be adjusted to a suitable angle.

The basic concepts have been described above, and it is apparent to those skilled in the art that the foregoing detailed disclosure is intended as an example only and does not constitute a limitation of the present disclosure. While not expressly stated herein, various modifications, improvements, and amendments may be made to the present disclosure by those skilled in the art. Those types of modifications, improvements, and amendments are suggested in the present disclosure, so those types of modifications, improvements, and amendments are still within the spirit and scope of the exemplary embodiments of the present disclosure.

What is claimed is:

1. A surgical robotic system for microwave waterjet, comprising a cutting blade, a serpentine joint pipe, a mechanical arm main body, a workbench, and a medical booster pump; wherein the cutting blade is connected to the mechanical arm main body through the serpentine joint pipe, the mechanical arm main body is electrically connected to the workbench, and the mechanical arm main body is connected to the medical booster pump via a pipeline, the cutting blade includes a casing, a microwave blade, a cooling support, a high-pressure jet pipe, and a return pipe, and each of two sides of a front end of the casing is provided with a protective plate, a front end of the microwave blade is provided with a curved cutting edge, the curved cutting edge is located at the front end of the casing and is located between the two protective plates, and a rear end of the microwave blade is provided in the cooling support, the casing is filled with a filler for fixing the cooling support, a tail end of the microwave blade is connected with a microwave conductor line, a jet hole is provided on one side of the curved cutting edge, the high-pressure jet pipe passes through the cooling support and is connected with the jet hole, and the return pipe passes through the cooling support and is provided on one side of the microwave blade.

2. The surgical robotic system of claim 1, wherein the cooling support includes an annular support and thermal insulation boards which are spaced apart from each other, the thermal insulation boards are provided in the annular support, the thermal insulation boards are provided with an isolation mounting groove, a first mounting hole, and a second mounting hole along a direction of the annular support, the rear end of the microwave blade is mounted in the isolation mounting groove, thermal conductive silicone is provided between the thermal insulation boards in the isolation mounting groove, the high-pressure jet pipe is provided in the first mounting hole, the return pipe is provided in the second mounting hole, and the filler is located between the thermal insulation boards that are spaced apart in each layer.

3. The surgical robotic system of claim 2, wherein an end of the isolation mounting groove is provided with a sealing ring for sealing the thermal conductive silicone.

4. The surgical robotic system of claim 1, wherein a cable shielding layer and an isolation layer are provided on a surface of the microwave conductor line sequentially from inside to outside.

5. The surgical robotic system of claim 1, wherein the jet hole coincides with an axis of the high pressure jet pipe.

6. The surgical robotic system of claim 1, wherein the serpentine joint pipe includes a flexible inner support pipe and a plurality of connecting pipe units fixed to the flexible inner support pipe at intervals, and the high pressure jet pipe, the return pipe, and the microwave conductor line are extended to an interior of the flexible inner support pipe, a front end of the flexible inner support pipe is fixedly connected to the casing, a rear end of the flexible inner support pipe is fixedly connected to the mechanical arm main body, a front end of each connecting pipe unit and a front end of the mechanical arm main body are symmetrically provided with two circular lugs, a rear end of the each connecting pipe unit and a rear end of the casing are symmetrically provided with two circular grooves cooperating with the two circular lugs, a line connecting the two circular lugs on the same connecting pipe unit is perpendicular to a line connecting the two circular grooves, and connectors and the connecting pipe units, two adjacent connecting pipe units, and the connecting pipe units and the mechanical arm main body are all spliced to each other by the circular lugs and the circular grooves.

7. The surgical robotic system of claim 6, wherein a plurality of traction ropes are fixedly connected along a circumferential direction to a rear end face of the casing, and a periphery of each of the connecting pipe units as well as a periphery of the mechanical arm main body are provided with a threading hole at a position corresponding to each of the traction ropes, and one end of each of the traction ropes away from the casing passes through the threading holes in the connecting pipe unit and in the mechanical arm main body sequentially.

8. The surgical robotic system of claim 6, wherein the flexible inner support pipe is a rubber pipe.
